(19) United States
(12) Patent Application Publication
(10) Pub. No.: US 2005/0137152 A1
(43) Pub. Date: Jun. 23, 2005

(54) Reovirus for the prevention of neoplasia

(76) Inventor: Danny Cheung, Calgary (CA)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(21) Appl. No.: 10/742,553

(22) Filed: Dec. 19, 2003

(51) Int. Cl. A61K 48/00

(52) U.S. Cl. 514/44; 424/93.21

(57) ABSTRACT

Methods for preventing pathogenic development of neoplasia, particularly Ras-mediated neoplasm, by administering reovirus to a mammal in need thereof, and use of reovirus for manufacture of a medicament for the prevention of neoplasia, are disclosed. The neoplasm is de novo. Human reovirus, non-human mammalian reovirus, and/or avian reovirus can be used. If the reovirus is human reovirus, type 1 (e.g., strain Lang), type 2 (e.g., strain Jones), type 3 (e.g., strain Dearing or strain Abney), as well as other serotypes or strains of reovirus can be used. A combination of different serotypes and/or different strains of reovirus, such as reovirus from different species of animal, can be used. The reovirus may be naturally occurring or modified. Preferably, the reovirus is modified.

### BACKGROUND OF THE INVENTION

1. Field of the Invention

The present invention pertains to methods for preventing pathogenic development of neoplasm, particularly Ras-mediated neoplasm, in a mammal in need thereof using reovirus.

2. References

U.S. Pat. No. 6,565,831 “Methods for preventing reovirus recognition for the treatment of cellular proliferative disorders”

U.S. Pat. No. 6,576,234 “Reovirus for the treatment of neoplasia”

U.S. Pat. No. 6,455,038 “Reovirus for the treatment of cellular proliferative disorders”

U.S. Pat. No. 6,344,195 “Reovirus for the treatment of neoplasia”

U.S. Pat. No. 6,261,555 “Reovirus for the treatment of neoplasia”

U.S. Pat. No. 6,136,307 “Reovirus for the treatment of cellular proliferative disorders”

U.S. Pat. No. 6,110,461 “Reovirus for the treatment of neoplasia”

U.S. Pat. No. 5,023,252 “Transdermal and trans-membrane delivery of drugs”

Chandron and Nibert, “Protease cleavage of reovirus capsid protein mu1 and mu1C is blocked by alkyl sulfate detergents, yielding a new type of infectious subvirion particle”, J. of Virology 72(1):467-75 (1998)

Gentsch, J. R. K. and Pacitti, A. F. (1985), J. Virol. 56:356

Lee, P. W. K. et al. (1999) Reovirus for the Treatment of Neoplasia, PCT International Application No. PCT/CA98/00774

Strong, J. E. and Lee, P. W. K., (1996) J. Virol., 70:612-616

Strong, J. E. et al. (1998), EMBO Vol. 17 No. 12 pp. 3351-3362

All of the above publications, patent applications and patents are herein incorporated by reference in their entirety to the same extent as if each individual publication, patent application or patent was specifically and individually indicated to be incorporated by reference in its entirety.

3. State of the Art

Normal cell proliferation is regulated by a balance between growth-promoting proto-oncogenes and growth-constraining tumor-suppressor genes. Tumorigenesis can be caused by genetic alterations to the genome that result in the mutation of those cellular elements that govern the interpretation of cellular signals, such as potentiation of proto-oncogene activity or inactivation of tumor suppression. It is believed that the interpretation of these signals ultimately influences the growth and differentiation of a cell, and that misinterpretation of these signals can result in neoplastic development and growth (neoplasia).

As previous study indicated, genetic alteration of the proto-oncogene Ras is believed to contribute to pathogenic development of approximately 30% of all human tumors (Wiessmuller, L. and Wittinghofer, F. (1994), Cellular Signaling 6(3):247-267; Barbacid, M. (1987) A Rev. Biochem. 56, 779-827).

As previous study indicated, the role that Ras plays in the pathogenesis of human tumors is specific to the type of tumor. Activating mutations in Ras itself are found in most types of human malignancies, and are highly represented in pancreatic cancer (80%), sporadic colorectal carcinomas (40-50%), human lung adenocarcinomas (15-24%), thyroid tumors (50%) and myeloid leukemia (30%) (Millis, N E et al. (1995) Cancer Res. 55:1444; Chaubert, P. et al. (1994), Am. J. Path. 144:767; Bos, J. (1989) Cancer Res. 49:4682). Ras activation is also demonstrated by upstream mitogenic signaling elements, notably by tyrosine receptor kinases (RTKs). These upstream elements, if amplified or overexpressed, ultimately result in elevated Ras activity by the signal transduction activity of Ras. Examples of this include overexpression of PDGFR in certain forms of glioblastomas, as well as in c-erbB-2/neu in breast cancer (Levitzki, A. (1994) Eur. J. Biochem. 226:1; James, P. W., et al. (1994) Oncogene 9:3601; Bos, J. (1989) Cancer Res. 49:4682).

As previous study indicated, reovirus is not known to be associated with any particular disease. Reovirus is a double-stranded RNA virus with a segmented genome. The receptor for the mammalian reovirus, sialic acid, is a ubiquitous molecule, therefore reovirus is capable of binding to a multitude of cells. However, most cells are not susceptible to reovirus infection and binding of reovirus to its cellular receptor results in no viral replication or virus particle production. This is probably the reason why reovirus is not known to be associated with any particular disease.

As recent study demonstrated, cells transformed with the ras oncogene become susceptible to reovirus infection, while their untransformed counterparts are not (Strong et al., 1998). For example, when reovirus-resistant NIH 3T3 cells were transformed with activated Ras or Sos, a protein which activates Ras, reovirus infection was enhanced. Similarly, mouse fibroblasts that are resistant to reovirus infection became susceptible after transfection with the EGF receptor gene or the v-erbB oncogene, both of which activate the ras pathway (Strong et al., 1996). Thus, reovirus can selectively infect and replicate in cells with an activated Ras pathway.

As recent study demonstrated, reovirus could be used as an oncolytic agent in a number of tumor bearing animal models (Lee, P. W. K. et al (1999)). Severe combined immune deficient mice (SCID) mice bearing tumors established from v-erbB-transformed murine NIH 3T3 cells or human U87 glioblastomas cells were treated with the virus (Coffey, M C et al 1998 Science 282: 1332). A single intratumoral injection of virus resulted in regression of tumor. Further, in animals given bilateral U87 tumor xenografts that a single unilateral injection of reovirus into the ipsilateral tumor resulted in reduction in the contralateral tumor. This reduction in the remote tumor site is the result of systemic spread of the reovirus. To examine the effect that a functional immune system could play upon this type of therapy, an immune competent mouse model was established. Treatment of immune-competent C3H mice bearing tumors established from ras-transformed C3H-10T1/2 cells also resulted in tumor regression.

Current methods of treatment for neoplasia include surgery, chemotherapy and radiation. Surgery is typically used as the primary treatment for early stages of cancer; however, many tumors cannot be completely removed by surgical means. In addition, metastatic growth of neoplasms may inhibit complete cure of cancer by surgery. Chemotherapy involves administration of compounds having antitumor activity, such as alkylating agents, antimetabolites, and antitumor antibiotics. The efficacy of chemotherapy is often limited by severe side effects, including nausea and vomiting, bone marrow depression, renal damage, and central nervous system depression. Radiation therapy relies on the greater ability of normal cells, in contrast with neoplastic cells, to repair themselves after treatment with radiation. Radiotherapy cannot be used to treat many neoplasms, however, because of the sensitivity of tissue surrounding the tumor. Clearly, there is a great need for improved therapies and, perhaps most important, a critical need for the prevention of the disease in the first instance (de novo, or primary prevention).

To date, there is no demonstrated, effective prevention therapy for de novo neoplasm. Further, there are no investigations in progress or contemplated for preventing neoplasm in the general population who are at no particular increased risk of developing neoplasm. Clearly, a great need exists for a neoplasm prevention therapy useful for the entire population, including individuals at high risk, as well as individuals at no particular increased risk, and including both men and women.

The current invention provides methods for the prevention of neoplasia.

### SUMMARY OF THE INVENTION

The present invention pertains to methods for preventing pathogenic development of neoplasia, particularly Ras-mediated neoplasm, in a mammal, using reovirus, and to use of reovirus for manufacture of a medicament for the prevention of neoplasia. Preferably, the reovirus may be chemically or biochemically pretreated to remove the outer coat or capsid and may thereby result in better infectivity of the virus. Preferably, the reovirus may be coated in a liposome or micelle to reduce or prevent an immune response from a mammal that has developed immunity to the reovirus and thus the existence of the reovirus in the mammal may be prolonged so as to yield a maximum result of prevention of neoplasia. When Ras signaling pathway is activated in a host cell, reovirus can infect and replicate in the cell thereby preventing pathogenic development of Ras-mediated neoplasm wherein the neoplasm is de novo. The reovirus can be administered to a mammal in need thereof. The reovirus can be administered in a single dose or in multiple doses. The reovirus can be administered intravenously, intraperitoneally, orally or intramuscularly into a mammal. The methods can be used to prevent neoplasia in a variety of mammals, including mice, dogs, cats, sheep, goats, cows, horses, pigs, and non-human primates. Preferably, the methods are used to prevent neoplasia in humans.

The methods and uses of the invention provide an effective means to prevent neoplasia.

Furthermore, because reovirus is not known to be associated with disease, any safety concerns associated with deliberate administration of a virus are minimized.

### DETAILED DESCRIPTION OF THE INVENTION

The present invention pertains to methods for preventing pathogenic development of neoplasm, particularly Ras-mediated neoplasm, in a mammal in need thereof using reovirus.

The name reovirus (Respiratory and enteric orphan virus) is a descriptive acronym suggesting that these viruses, although not associated with any known disease state in humans, can be isolated from both the respiratory and enteric tracts (Sabin, A. B. (1959), Science 130:966). The term “reovirus” refers to all viruses classified in the reovirus genus.

Reoviruses are viruses with a double-stranded, segmented RNA genome. The virions measure 60-80 nm in diameter and possess two concentric capsid shells, each of which is icosahedral. The genome consists of double-stranded RNA in 10-12 discrete segments with a total genome size of 16-27 kbp. The individual RNA segments vary in size. Three distinct but related types of reovirus have been recovered from many species. All three types share a common complement-fixing antigen.

The human reovirus consists of three serotypes: type 1 (strain Lang or T1L), type 2 (strain Jones, T2J) and type 3 (strain Dearing or strain Abney, T3D). The three serotypes are easily identifiable on the basis of neutralization and hemagglutinin-inhibition assays (Sabin, A. B. (1959), Science 130:966; Fields, B. N. et al. (1996), Fundamental Virology, 3rd Edition, Lippincott-Raven; Rosen, L. (1960) Am. J. Hyg. 71:242; Stanley, N. F. (1967) Br. Med. Bull. 23:150).

Although reovirus is not known to be associated with any particular disease, many people have been exposed to reovirus by the time they reach adulthood (i.e., fewer than 25% in children<5 years old, to greater than 50% in those 20-30 years old (Jackson G. G. and Muldoon R. L. (1973) J. Infect. Dis. 128:811; Stanley N. F. (1974) In: Comparative Diagnosis of Viral Diseases, edited by E. Kurstak and K. Kurstak, 385-421, Academic Press, New York).

For mammalian reoviruses, the cell surface recognition signal is sialic acid (Armstrong, G. D. et al. (1984), Virology 138:37; Gentsch, J. R. K. and Pacitti, A. F. (1985), J. Virol. 56:356; Paul R. W. et al. (1989) Virology 172:382-385). Due to the ubiquitous nature of sialic acid, reovirus binds efficiently to a multitude of cell lines and as such can potentially target many different tissues; however, there are significant differences in susceptibility to reovirus infection between cell lines. This is probably the reason why reovirus is not known to be associated with any particular disease.

Prior to describing the invention in further detail, the terms used in this application are defined as follows unless otherwise indicated.

### Definitions

The term “prevent”, when used in conjunction with neoplasm, includes reducing the likelihood of a mammal or human incurring or developing neoplasm. The term does not include treating a mammal or human diagnosed with neoplasm.

The term “de novo”, as used in the current invention, means the lack of transformation or metamorphosis of normal cells to cancerous or malignant cells in the first instance. Such a transformation may occur in stages in the same or daughter cells via an evolutionary process or may occur in a single, pivotal event. This de novo process is a process distinct from that of metastasis, colonization, or spreading of already transformed or malignant cells from the primary tumor site to new locations. The term “de novo” is associated with primary prevention. This invention also relates to the administration of a reovirus to a mammal or human who is at increased risk of developing neoplasm.

A mammal or human who is at no particular risk of developing neoplasm is one who may develop de novo neoplasm, has no evidence or suspicion of the potential of the disease above normal risk, and who has never had a diagnosis of having the disease. The increased risk factor contributing to the development of neoplasm is a history of neoplasm in a mammal or human, even when there is no evidence of residual disease. Another well-accepted risk factor is family history of the disease.

The term “a mammal in need thereof” refers to a mammal or human who will benefit from the method of this invention. The mammal or human is believed to be either at increased risk or at no particular risk of developing neoplasm. The term does not include treating a mammal or human diagnosed with neoplasm.

The term “reovirus” refers to any virus classified in the reovirus genus, whether naturally occurring, modified or recombinant. Reoviruses are viruses with a double-stranded, segmented RNA genome. The virions measure 60-80 nm in diameter and possess two concentric capsid shells, each of which is icosahedral. The genome consists of double-stranded RNA in 10-12 discrete segments with a total genome size of 16-27 kbp. The individual RNA segments vary in size. Three distinct but related types of reovirus have been recovered from many species. All three types share a common complement-fixing antigen. The human reovirus consists of three serotypes: type 1 (strain Lang or T1L), type 2 (strain Jones, T2J) and type 3 (strain Dearing or strain Abney, T3D). The three serotypes are easily identifiable on the basis of neutralization and hemagglutinin-inhibition assays (see, for example, Fields, B. N. et al., 1996).

The reovirus may be naturally occurring or modified. The reovirus is “naturally-occurring” when it can be isolated from a source in nature and has not been intentionally modified by humans in the laboratory. For example, the reovirus can be from a “field source”, that is, from a human who has been infected with the reovirus.

The reovirus may be modified such that the outer capsid is removed, the virion is packaged in a liposome or micelle or the proteins of the outer capsid have been mutated.

The reovirus may be a recombinant (i.e. reasserted) reovirus from two or more types of reoviruses with differing pathogenic phenotypes such that it contains different antigenic determinants, thereby reducing or preventing an immune response by a mammal previously exposed to a reovirus subtype. Such recombinant virions can be generated by co-infection of mammalian cells with different subtypes of reovirus with the resulting resorting and incorporation of different subtype coat proteins into the resulting virion capsids.

The term “neoplasm” refers to an abnormal tissue growth, generally forming a distinct mass, that grows by cellular proliferation more rapidly than normal tissue growth. Neoplasms show partial or total lack of structural organization and functional coordination with normal tissue. These can be broadly classified into three major types. Malignant neoplasms arising from epithelial structures are called carcinomas, malignant neoplasms that originate from connective tissues such as muscle, cartilage, fat or bone are called sarcomas and malignant tumors affecting hematopoietic structures (structures pertaining to the formation of blood cells) including components of the immune system, are called leukemias and lymphomas. A tumor is the neoplastic growth of the disease cancer. As used herein, a “neoplasm”, also referred to as a “tumor”, is intended to encompass hematopoietic neoplasms as well as solid neoplasms.

The term “Ras-mediated neoplasm” means that at least some of the cells of the neoplasm have a mutation in which the Ras gene (or an element of the Ras signaling pathway) is activated, either directly (e. g., by an activating mutation in Ras) or indirectly (e. g., by activation of an upstream element in the Ras pathway). Activation of an upstream element in the Ras pathway includes, for example, transformation with epidermal growth factor receptor (EGFR) or Sos. A neoplasm that results, at least in part, by the activation of Ras, an upstream element of Ras, or an element in the Ras signalling pathway is referred to herein as a “Ras-mediated neoplasm”.

### Methods

In the methods of the invention, reovirus is administered to a mammal in need thereof for preventing pathogenic development of neoplasm, particularly Ras-mediated neoplasm. Representative types of human reovirus that can be used include type 1 (e.g., strain Lang or T1L); type 2 (e.g., strain Jones or T2J); and type 3 (e.g., strain Dearing or strain Abney, T3D or T3A); other strains of reovirus can also be used. In a preferred embodiment, the reovirus is human reovirus serotype 3, more preferably the reovirus is human reovirus serotype 3, strain Dearing. Alternatively, the reovirus can be a non-human mammalian reovirus (e.g., non-human primate reovirus, such as baboon reovirus; equine; or canine reovirus), or a non-mammalian reovirus (e.g., avian reovirus). A combination of different serotypes and/or different strains of reovirus, such as reovirus from different species of animal, can be used.

The reovirus may be naturally occurring or modified. The reovirus is “naturally-occurring”: when it can be isolated from a source in nature and has not been intentionally modified by humans in the laboratory. For example, the reovirus can be from a “field source”: that is, from a human patient.

The reovirus may be modified but still capable of infecting a mammalian cell having an active ras pathway. The reovirus may be chemically or biochemically pretreated (e.g., by treatment with a protease, such as chymotrypsin or trypsin) prior to administration to a mammal. Pretreatment with a protease removes the outer coat or capsid of the virus and may increase the infectivity of the virus. The reovirus may be coated in a liposome or micelle (Chandron and Nibert, “Protease cleavage of reovirus capsid protein mu1 and mu1C is blocked by alkyl sulfate detergents, yielding a new type of infectious subvirion particle”, J. of Virology 72(1):467-75 (1998)) to reduce or prevent an immune response from a mammal which has developed immunity to the reovirus. For example, the virion may be treated with chymotrypsin in the presence of micelle forming concentrations of alkyl sulfate detergents to generate a new infectious subvirion particle.

The reovirus is preferably a reovirus modified to reduce or eliminate an immune reaction to the reovirus. Such modifications could include packaging of the reovirus in a liposome, a micelle or other vehicle to mask the reovirus from the mammals immune system. Alternatively, the outer capsid of the reovirus virion particle may be removed since the proteins present in the outer capsid are the major determinant of the host humoral and cellular responses.

Preferably, the reovirus may be chemically or biochemically pretreated to remove the outer coat or capsid and may thereby result in better infectivity of the virus. Preferably, the reovirus may be coated in a liposome or micelle to reduce or prevent an immune response from a mammal that has developed immunity to the reovirus and thus the existence of the reovirus in the mammal may be prolonged so as to yield a maximum result of prevention of neoplasia. When Ras signaling pathway is activated in a host cell, reovirus can infect and replicate in the cell thereby preventing pathogenic development of Ras-mediated neoplasm wherein the neoplasm is de novo.

The reovirus may be a recombinant reovirus from two or more types of reoviruses with differing pathogenic phenotypes such that it contains different antigenic determinants thereby reducing or preventing an immune response by a mammal previously exposed to a reovirus subtype. Such recombinant virions, also known as reassortants, can be generated by co-infection of mammalian cells with different subtypes of reovirus with the resulting resorting and incorporation of different subtype coat proteins into the resulting virion capsids.

The reovirus may be modified by incorporation of mutated coat proteins, such as for example .sigma. 1, into the virion outer capsid. The proteins may be mutated by replacement, insertion or deletion. Replacement includes the insertion of different amino acids in place of the native amino acids. Insertions include the insertion of additional amino acid residues into the protein at one or more locations. Deletions include deletions of one or more amino acid residues in the protein. Such mutations may be generated by methods known in the art. For example, oligonucleotide site directed mutagenesis of the gene encoding for one of the coat proteins could result in the generation of the desired mutant coat protein. Expression of the mutated protein in reovirus infected mammalian cells in vitro such as COS1 cells will result in the incorporation of the mutated protein into the reovirus virion particle (Turner and Duncan, “Site directed mutagenesis of the C-terminal portion of reovirus protein sigmal: evidence for a conformation-dependent receptor binding domain” Virology 186(1):219-27 (1992); Duncan et al., “Conformational and functional analysis of the C-terminal globular head of the reovirus cell attachment protein” Virology 182(2):810-9 (1991); Mah et al., “The N-terminal quarter of reovirus cell attachment protein sigma 1 possesses intrinsic virion-anchoring function” Virology 179(1):95-103 (1990)).

The reovirus can be administered to a mammal in need thereof. Representative mammals include mice, dogs, cats, sheep, goats, cows, horses, pigs, non-human primates, and humans. In a preferred embodiment, the mammal is a human.

One neoplasm that is particularly susceptible thereto by the methods of the invention is pancreatic cancer, because of the prevalence of Ras-mediated neoplasms associated with pancreatic cancer. Other neoplasms that are particularly susceptible thereto by the methods of the invention include breast cancer, central nervous system cancer (e.g., neuroblastoma and glioblastoma), peripheral nervous system cancer, lung cancer, prostate cancer, colorectal cancer, thyroid cancer, renal cancer, adrenal cancer, liver cancer, and leukemia.

The route by which the reovirus is administered, as well as the formulation, carrier or vehicle, will depend on the type of the neoplasm to prevent. A wide variety of administration routes can be employed. For example, for a hematopoietic neoplasm, the reovirus can be administered intravenously or intravascularly. For neoplasms that are not easily accessible within the body, such as brain tumors, the reovirus is administered in a manner such that it can be transported systemically through the body of the mammal (e.g., intrathecally, intravenously or intramuscularly). The reovirus can also be administered subcutaneously, intraperitoneally, topically (e.g., for melanoma), orally (e.g., for oral or esophageal neoplasm), rectally (e.g., for colorectal neoplasm), vaginally (e.g., for cervical or vaginal neoplasm), nasally or by inhalation spray (e.g., for lung neoplasm).

This invention also includes pharmaceutical compositions which contain, as the active ingredient, one or more of the reoviruses associated with “pharmaceutically acceptable carriers or excipients”. In making the compositions of this invention, the active ingredients/reovirus is usually mixed with an excipient, diluted by an excipient or enclosed within such a carrier which can be in the form of a capsule, sachet, paper or other container. When the pharmaceutically acceptable excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

For preparing solid compositions such as tablets, the principal active ingredients/reovirus is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the novel compositions of the present invention may be incorporated for administration orally or by injection include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described herein. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices which deliver the formulation in an appropriate manner.

Another preferred formulation employed in the methods of the present invention employs transdermal delivery devices (“patches”). Such transdermal patches may be used to provide continuous or discontinuous infusion of the reovirus of the present invention in controlled amounts. The construction and use of transdermal patches for the delivery of pharmaceutical agents is well known in the art. See, for example, U.S. Pat. No. 5,023,252, herein incorporated by reference. Such patches may be constructed for continuous, pulsatile, or on demand delivery of pharmaceutical agents.

Other suitable formulations for use in the present invention can be found in Remington's Pharmaceutical Sciences.

The reovirus is administered in an amount that is sufficient to prevent a Ras-mediated neoplasm (e.g., an “effective amount”). Preferably the effective amount is that amount able to prevent neoplasm. Preferably the effective amount is from about 1.0 pfu/kg body weight to about 10.sup.15 pfu/kg body weight, more preferably from about 10.sup.2 pfu/kg body weight to about 10.sup.13 pfu/kg body weight. For example, for prevention of a human, approximately 10.sup.2 to 10.sup.15 plaque forming units (PFU) of reovirus can be used. The effective amount will be determined on an individual basis and may be based, at least in part, on consideration of the type of reovirus; the chosen route of administration; the individual's size, age, gender; and the like.

The reovirus can be administered in a single dose, or multiple doses (i.e., more than one dose). The multiple doses can be administered concurrently, or consecutively (e.g., over a period of days, weeks or months).

The compositions are preferably formulated in a unit dosage form, each dosage containing an appropriate amount from about 10.sup.2 pfus to about 10.sup.15 pfus of the reovirus. The term “unit dosage forms” refers to physically discrete units suitable as unitary dosages for human subjects and other mammals, each unit containing a predetermined quantity of reovirus calculated to produce the desired prevention effect, in association with a suitable pharmaceutical excipient.

1. Use of a reovirus for the manufacture of a medicament for preventing a Ras-mediated neoplasm in a mammal.

2. The use of claim 1, wherein the reovirus is a human reovirus.

3. The use of claim 2, wherein the human reovirus is selected from the group consisting of: type 1 reovirus, type 2 reovirus, and type 3 reovirus.

4. The use of claim 1, wherein the reovirus is a non-human reovirus.

5. The use of claim 4, wherein the non-human reovirus is selected from the group consisting of: mammalian reovirus and avian reovirus.

6. The use of claim 1, wherein more than one type of reovirus is administered.

7. The use of claim 1, wherein more than one strain of reovirus is administered.

8. The use of claim 1, wherein the reovirus is a field isolate.

9. The use of claim 1, wherein the reovirus is treated with a protease prior to administration.

10. The use of claim 1, wherein the reovirus is encapsulated in a micelle.

11. The use of claim 1, wherein the mammal is selected from the group consisting of: mice, dogs, cats, sheep, goats, cows, horses, pigs, and non-human primates.

12. The use of claim 1, wherein the mammal is a human.

13. The use of claim 1, wherein the neoplasm is selected from the group consisting of: pancreatic cancer, breast cancer, central nervous system cancer, and peripheral nervous system cancer.

14. The use of claim 1, wherein the neoplasm is selected from the group consisting of: lung cancer, prostate cancer, colorectal cancer, thyroid cancer, renal cancer, adrenal cancer, liver cancer, and leukemia.

15. The use of claim 1, wherein the ras-mediated neoplasm is de novo.

16. A method of preventing a Ras-mediated neoplasm in a mammal in need thereof, comprising administering to the mammal an effective amount of a reovirus.

17. The method of claim 16, wherein the reovirus is a human reovirus.

18. The method of claim 17, wherein the human reovirus is selected from the group consisting of: type 1 reovirus, type 2 reovirus, and type 3 reovirus.

19. The method of claim 16, wherein the reovirus is a non-human reovirus.

20. The method of claim 19, wherein the non-human reovirus is selected from the group consisting of: mammalian reovirus and avian reovirus.

21. The method of claim 16, wherein more than one type of reovirus is administered.

22. The method of claim 16, wherein more than one strain of reovirus is administered.

23. The method of claim 16, wherein the reovirus is a field isolate.

24. The method of claim 16, wherein the reovirus is treated with a protease prior to administration.

25. The method of claim 16, wherein the reovirus is encapsulated in a micelle.

26. The method of claim 16, wherein the reovirus is administered intravenously into the mammal.

27. The method of claim 16, wherein the reovirus is administered intraperitoneally into the mammal.

28. The method of claim 16, wherein the reovirus is administered intramuscularly into the mammal.

29. The method of claim 16, wherein the reovirus is administered orally.

30. The method of claim 16, wherein the mammal is selected from the group consisting of: mice, dogs, cats, sheep, goats, cows, horses, pigs, and non-human primates.

31. The method of claim 16, wherein the mammal is a human.

32. The method of claim 16, wherein the neoplasm is selected from the group consisting of: pancreatic cancer, breast cancer, central nervous system cancer, and peripheral nervous system cancer.

33. The method of claim 16, wherein the neoplasm is selected from the group consisting of: lung cancer, prostate cancer, colorectal cancer, thyroid cancer, renal cancer, adrenal cancer, liver cancer, and leukemia.

34. The method of claim 16, wherein approximately 1 to 10.sup.15 plaque forming units of reovirus/kg body weight are administered.

35. The method of claim 16, wherein the reovirus is administered in a single dose.

36. The method of claim 16, wherein the reovirus is administered in more than one dose.

37. The method of claim 16, wherein the ras-mediated neoplasm is de novo.

38. A method of preventing a Ras-mediated neoplasm in a human in need thereof, comprising administering to the human an effective amount of a reovirus.
